SEQUENCE LISTING (3 sequences)

SEQ ID NO: 1 - protein, 440 aa, unidentified
MAREPTALATAAAIRSGATTARAETEAAIARIEALDGAINAVVVRDFDRALAAADAADARIQAGDTAPLLGVPMTVKEAFDVEGLPTHWGFRQHAGNIATSDAEAVRRLKAAGAIILGKTNVPKGLGDWQSVNSIHGVTNHPLDPTRTPGGSSGGSAAALASGMVPLELGSDIGGSIRIPAHFCGVWGLKPSWGAISSHGHRYPGTNGAETPLGVIGPMARSPDDLAAMLDLLATLPMPRASRPPRRVLAITDHPAIRTSAVCRDAVDTAAEALAGAGIEVIRSTDLLPDLARQHHAYGQMLSVAFARSDPTLHASLPNLLTWLSWQDAQARNTRAWGRLFGEVDAVIAPPAATQAFAHDHAPQANRTLDIDGVASPYDAHLAWAGVATYPGLPAVVVPVGTANGLPVGVQVITDFHRDHDAIATAALIHRLTEGQPALE

SEQ ID NO: 2 - protein, 441 aa, unidentified
MSELSAIETAAAIAGGSMTALEACDAAIARIEQRDGPINAVVVRDFDRARDAAKAADAEIAAAVRKPLLGVPMTIKESFDIAGLPTSWGFAEHADHIATADSLVVSRLKAAGAVFLGKSNIPVGLADWQSVNPNYGRTNNPHDHSRSAGGSSGGAAAALAAGMVPLEYGSDIGGSIRVPAHFCGVWGLKTTFDAVSLEGHYFPRTDSAKADLSVVGPMARTPADLALALDITSKVPLPQSRIANLSGLRILLLTAHPETVADSATISAVERAAAACEASGATVATSSPDLPDLSALVADYTRMLLVVLARGLAPEGTEPVSLNAWYAMLDDQARMMRAFDRLFESFDAIFCPVLGTTAFAHSDEPDWAKRSLSIDGGIAPFAAQLGWISMATYGGMPALSMPLGADGNGLPINLQIITRNWSDHDAIRIGALVAEALDRLE

SEQ ID NO: 3 - protein, 441 aa, unidentified
MSELSAIETAAAIAGGSMTALEACDAAIARIEQRDGPINAVVVRDFDRAREAAKAADGEVAAGVSKPLLGVPMTIKESIDIAGLPTSWGFAEHADHIATADSVVVSRLKAAGAVFLGKTNIPVALADWQSSNPNYGRTNNPHDLTRSAGGSSGGAAAALAAGMVPLEYGSDIGGSIRVPAHFCGVWGLKTTFDAVSLEGHYLPRTDGARGELGVVGPMARNPQDLALALDLTSRIALPIARIDTLNGLRILLLTHHPRAAADSAVVAAVEKAAESCAAQGAQVSTSNADLPDLSKLVSDYTRMLLIVLAQGKAPEGTEPVSLNAWYGMLDDQARTIRGFDRLFDSFDAIFCPVLGTSAFPHSDEADWGKRTLTIDGADTPFGSQLAWISMATYCGMPALSMPVGTDANGLPIGLQIITRNWSDHDAVRIGALVADALAALE

(19) United States
(12) Patent Application Publication
(10) Pub. No.: US 2026/0055254 A1
(43) Pub. Date: Feb. 26, 2026

(54) NOVEL URETHANASES FOR THE ENZYMATIC DEGRADATION OF POLYURETHANES

(71) Applicant: Covestro Deutschland AG, Leverkusen (DE)

(72) Inventors: Lukas Reisky, Köln (DE); Gernot Jaeger, Rösrath (DE); Simone Goebbels, Korschenbroich (DE); Vera Essmann, Köln (DE); Igor Pochorovski, Monheim am Rhein (DE); Uwe Bornscheuer, Greifswald (DE); Christoffel Badenhorst, Greifswald (DE); Ren Wei, Greifswald (DE); Yannick Branson, Greifswald (DE); Carolin Buchmann, Greifswald (DE)

(21) Appl. No.: 18/853,763

(22) PCT Filed: Apr. 5, 2023
(86) PCT No.: PCT/EP2023/058947

§ 371 Date: Oct. 3, 2024

(30) Foreign Application Priority Data
Apr. 8, 2022 (EP) 22167501.0
Apr. 8, 2022 (EP) 22167503.6
Apr. 8, 2022 (EP) 22167505.1

(51) Int. Cl. C08J 11/10; C12N 9/80; C12P 7/18; C12P 13/00

(52) U.S. Cl. CPC C08J 11/105; C12N 9/80; C12P 7/18; C12P 13/001; C12Y 305/01075

(57) ABSTRACT

The invention relates to novel urethanases for the enzymatic degradation of polyurethanes and to an enzymatic process for the complete degradation of polyurethanes in defined monomers.

The present invention relates to new urethanases for the enzymatic breakdown of polyurethanes and to an enzymatic process for the complete breakdown of polyurethanes into defined monomers.

Polyurethanes are established in many areas of normal life. They can be found, for example, in soft foams (mattresses, sponges, upholstered furniture), rigid foams (insulation materials, building materials), thermoplastics (sports shoes) or coatings (varnishes, paints, adhesives). The constantly increasing demand for products means that ever greater volumes are being produced. At the same time, there is a growing need for methods that maximize the sustainable recycling of polyurethane products that are no longer needed and so allow the building blocks of the polymers to be reused. For this, the bonds in the polyurethanes must be selectively cleaved in order to be able to obtain defined breakdown products, thereby making them recyclable.

In addition to the physiological functions that enzymes perform in living organisms, enzymes can be used in a diversity of ways for the catalysis of chemical reactions outside this context. Such reactions can be carried out under milder conditions than conventional chemical processes, for example lower temperature, neutral pH, and without the use of aggressive chemicals. Through this it is possible to save on energy, minimize the formation of by-products, and protect the environment, which helps to reduce operating costs. In some cases, it is only through the use of enzymes that it is possible for labile starting materials to be used as reaction feedstocks (Jaeger, K.-E. & Reetz, M. T. (1998) Microbial lipases form versatile tools for biotechnology. Trends in biotechnology, 16, 396-403). Moreover, enzymes are often chemo-, regio-, stereo- and enantioselective, which makes the purification of the products substantially easier, which can permit the efficient synthesis of products that are otherwise difficult to obtain (Hasan, F., Shah, A. A. & Hameed, A. (2006) Industrial applications of microbial lipases. Enzyme and Microbial Technology, 39, 235-251).

There are nowadays only very few cases of recycling of polyurethanes. Disposal involves landfill or else complete incineration, and although energy is obtained in the case of the latter, there is no efficient reuse of the polymer building blocks.

It is known that polyurethanes can be broken down to a certain degree by bacteria and fungi. Polyester polyurethanes are considerably more susceptible to such microbial/enzymatic breakdown than polyether polyurethanes (Nakajima-Kambe, T., Shigeno-Akutsu, Y., Nomura, N., Onuma, F. & Nakahara, T. (1999) Microbial degradation of polyurethane, polyester polyurethanes and polyether polyurethanes. Applied microbiology and biotechnology, 51, 134-140).

The breakdown of polyester polyurethanes can be readily accomplished by hydrolysis of the ester linkages. The relatively simple breakdown of polyesters is not surprising, given that ester linkages in hydrophobic substrates in nature must also be cleaved when lipids are broken down and polyesters without urethane linkages can likewise be broken down relatively easily by esterases and lipases (Marten, E., Müller, R.-J. & Deckwer, W.-D. (2003) Studies on the enzymatic hydrolysis of polyesters I. Low molecular mass model esters and aliphatic polyesters. Polymer degradation and stability, 80, 485-501; Marten, E., Müller, R.-J. & Deckwer, W.-D. (2005) Studies on the enzymatic hydrolysis of polyesters. II. Aliphatic-aromatic copolyesters. Polymer degradation and stability, 88, 371-381.). Enzymes used to break down polyurethane have been characterized as esterases in various literature sources (Allen, A. B., Hilliard, N. P. & Howard, G. T. (1999) Purification and characterization of a soluble polyurethane degrading enzyme from Comamonas acidovorans. International biodeterioration & biodegradation, 43, 37-41; Blake, R., Norton, W. & Howard, G. (1998) Adherence and growth of a Bacillus species on an insoluble polyester polyurethane. International biodeterioration & biodegradation, 42, 63-73; Crabbe, J. R., Campbell, J. R., Thompson, L., Walz, S. L. & Schultz, W. W. (1994) Biodegradation of a colloidal ester-based polyurethane by soil fungi. International biodeterioration & biodegradation, 33, 103-113; Darby, R. T. & Kaplan, A. M. (1968) Fungal susceptibility of polyurethanes. Applied microbiology, 16, 900-905; Howard, G. T., Norton, W. N. & Burks, T. (2012) Growth of Acinetobacter gerneri P7 on polyurethane and the purification and characterization of a polyurethanase enzyme. Biodegradation, 23, 561-573; Kaplan, A. M., Darby, R. T., Greenberger, M. & Rodgers, M. (1968) Microbial deterioration of polyurethane systems. Dev Ind Microbiol, 82, 362-371; Kay, M., Morton, L. & Prince, E. (1991) Bacterial degradation of polyester polyurethane. International biodeterioration, 27, 205-222; Vega, R. E., Main, T. & Howard, G. T. (1999) Cloning and expression in Escherichia coli of a polyurethane-degrading enzyme from Pseudomonas fluorescens. International biodeterioration & biodegradation, 43, 49-55). There is no clear demonstration therein of cleavage of the urethane linkage, since there were no instances of enzyme characterization being carried out on the basis of cleavage of a molecule having a urethane group.

The breakdown of poly(ester urethane) s with fungi or bacteria is described in many publications and patents. However, the breakdown mostly targets only the relatively easily cleaved ester linkages and is mostly demonstrated only by macroscopic observation of polymer breakdown. There is no controlled breakdown here of ester and urethane linkages as in the present invention, and long breakdown times often result. These publications show that urethanases are commonly found enzymes, but provide no demonstration of the specific capabilities, potential uses, and grouping thereof, as employed in the present invention. (JP09192633, Tang, Y. W., Labow, R. S., Santerre, J. P. (2003) Enzyme induced biodegradation of polycarbonate-polyurethanes: dose dependence effect of cholesterol esterase. Biomaterials 24 (12), 2003-2011, Vega, R. E., Main, T. & Howard, G. T. (1999) Cloning and expression in Escherichia coli of a polyurethane-degrading enzyme from Pseudomonas fluorescens. International biodeterioration & biodegradation, 43, 49-55)

A breakdown process for the enzymatic breakdown of poly(ester urethane) s is known, the first step of which is to obtain an esterase from a culture of Comamonas acidovorans by using only poly(ester urethane) as the carbon source. In a complicated purification step, the esterase is separated and used for the breakdown of poly(ester urethane) s in a batch process. This gives rise to long breakdown times in a multistage process and no demonstration of specific cleavage of the urethane linkages (JP 09201192, JP 10271994).

The breakdown of poly(ester urethane) s with cutinases, esterases, and/or lipases is described in various patents and publications. However, the breakdown here as well targets only the relatively simple cleavage of the ester linkages, but not specifically the urethane linkages. In addition, no specific combination of enzymes that cleave ester and urethane linkages is described for the selective control of breakdown. It can be assumed that the described processes result in little or no cleavage of the urethane linkage. This means that diamines used in the synthesis of polyurethanes cannot be recovered efficiently. (EP 0968300, U.S. Pat. No. 6,180,381)

WO 2013/134801 describes the breakdown of aromatic polyurethanes based on polyether polyols using an enzyme of class EC 3. No specific enzyme sequences are stated, consequently neither the specificity of the process in the breakdown of particular urethane linkages, nor the controlled cleavage of ester linkages and separate cleavage of urethane linkages, as shown in the present invention, are demonstrated in the cited patent.

WO 2006/019095 describes a urethanase and variants of this enzyme obtained by protein engineering. The enzyme can cleave urethane oligomers based on TDA or MDA. Furthermore, WO 2019/243293 describes newly identified urethanases.

It was thus an object of the present invention to provide further enzymes that can be used for the enzymatic cleavage of urethane linkages and preferably for the complete enzymatic breakdown of polyurethanes. A further object was to provide an enzymatic process that allows the breakdown of polyurethanes into defined monomers.

This object is achieved by the embodiments disclosed in the claims and in the description below.

In a first embodiment, the present invention relates to a polypeptide having an amino acid sequence as defined by SEQ ID NO.: 1, 2 or 3, or a variant thereof, wherein the variant is obtained by the addition, deletion or exchange of up to 15% of the amino acids present in the respective polypeptide defined by SEQ ID NO.: 1, 2 or 3, characterized in that the polypeptide has urethanase activity.

Preferably, a polypeptide having a sequence according to SEQ ID NO.: 1, 2 or 3 consists of the amino acid sequence defined by SEQ ID NO.: 1, 2 or 3.

### Polypeptide

The term “polypeptide” is well known to those skilled in the art. It refers to a chain of at least 50, preferably at least 70, amino acids linked to one another by peptide bonds. A polypeptide may comprise both naturally occurring and synthetic amino acids. It preferably comprises the known proteinogenic amino acids and also optionally selenocysteine, pyrrolysine and hydroxyproline. It more preferably consists of the known proteinogenic amino acids and also optionally additionally of selenocysteine, pyrrolysine and hydroxyproline.

A variant is obtained from the amino acid sequences according to the invention defined by SEQ ID NO.: 1, 2 or 3 preferably by adding, deleting or exchanging up to 10%, and more preferably up to 5%, of the amino acids present in the respective polypeptide. The basis for calculating the sequence identity is preferably in each case the amino acid sequence defined by SEQ ID NO.: 1, 2 or 3. It is known to those skilled in the art to form fusion proteins of enzymes with other proteins without this affecting the activity of the enzyme. Therefore, the term “variant” in a further embodiment of the invention also includes amino acid sequences derived from the polypeptide defined by SEQ ID NO.: 1, 2 or 3 that are fused at the N-terminus and/or at the C-terminus with other proteins, for example green fluorescent protein.

Particularly preferred variants of the polypeptides according to the invention are obtained by adding, deleting or exchanging up to 20, preferably up to 10, and more preferably up to 5, amino acids of the disclosed sequences. The aforementioned modifications may in principle be made continuously or discontinuously at any desired point in the polypeptide in question. However, they are preferably made solely at the N-terminus and/or at the C-terminus of the polypeptide. Each variant according to the invention obtained by adding, exchanging or deleting amino acids is, however, characterized by urethanase activity as defined in this application hereinbelow.

### Urethanase Activity

The term “urethanase activity” refers to the ability of a polypeptide to enzymatically catalyze the cleavage of a urethane group. In this process, each mole of urethane group gives rise to one mole of amine, one mole of alcohol, and one mole of CO₂.

The urethane group may be an aromatically, cycloaliphatically or aliphatically attached urethane group. In the case of an aromatically attached urethane group, the nitrogen atom is attached directly to an aromatic ring. In the case of an aliphatically attached urethane group, the nitrogen atom is attached to an alkyl radical that is part of an open-chain alkyl radical. The alkyl radical is preferably unbranched and composed of at least one, preferably at least two, and more preferably at least three, carbon atoms. In the case of a cycloaliphatically attached urethane group, the nitrogen atom is attached to a carbon atom that is part of an aliphatic ring. This ring may be unsaturated in one or more positions, provided it does not acquire aromatic character as a result of the presence of double bonds. In a preferred embodiment of the present invention, the polypeptide having urethanase activity is capable of enzymatically cleaving an aromatically attached urethane group.

Whether a polypeptide has urethanase activity can be checked through the cleavage of suitable model substrates. They are preferably the substrates shown to be cleaved in the working examples.

For the ability to cleave aromatically attached urethane groups, the model substrates particularly preferably used are ethyl 4-nitrophenyl carbamate (ENPC) or the carbamate obtainable by the reaction of 7-amino-4-methylcoumarin with ethyl chloroformate (EMACC). The cleavage of ENPC is detected by determining the increase in the concentration of 4-nitroaniline. This is done preferably photometrically at a wavelength of 405 nm. The enzyme activity is determined preferably in a reaction buffer containing 100 mM of K₂HPO₄/KH₂PO₄, PH 7 with 6.25% by volume of ethanol in the presence of 0.2 mg/L of ENPC as substrate. Incubation of the enzyme with ENPC in the reaction buffer is carried out preferably at room temperature and preferably for 24 hours.

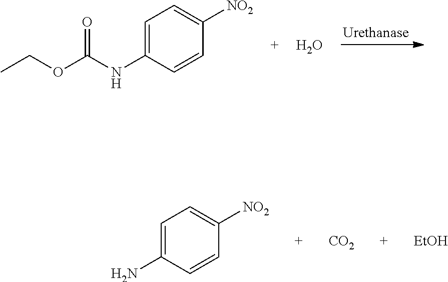

The cleavage of EMACC is detected by determining the increase in the concentration of 7-amino-4-methylcoumarin (AMC). This is done preferably photometrically at a wavelength of 365 nm. The enzyme activity is determined preferably in a reaction buffer containing 50 mM of Na₂HPO₄/NaH₂PO₄, PH 8.0 with 0.2% by volume of DMSO in the presence of 100 μM of EMACC as substrate. Incubation of the enzyme with EMACC in the reaction buffer is carried out preferably at 30° C. and preferably for up to 24 hours.

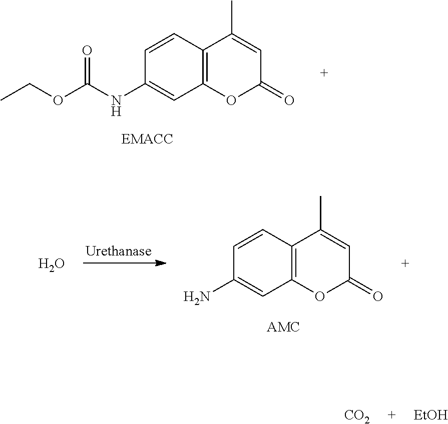

The model substrate for the ability to cleave aliphatically attached urethane groups is preferably ethyl phenethyl carbamate (EPEC). Cleavage is detected by determining the increase in the concentration of phenethylamine. This is done preferably by HPLC. The reaction buffer used and the reaction conditions preferably correspond to the parameters described above for ENPC.

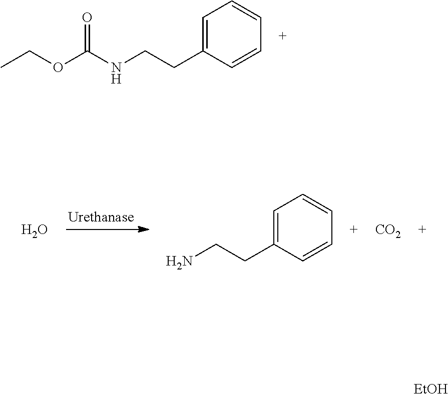

The urethane linkages which can be cleaved by the urethanases according to the invention are formally based on the addition of a polyisocyanate having aliphatically, cycloaliphatically, araliphatically or aromatically attached isocyanate groups and an alcohol.

In an isocyanate having aliphatically attached isocyanate groups, all isocyanate groups are attached to a carbon atom that is part of an open carbon chain. This may be unsaturated in one or more positions. The aliphatically attached isocyanate group or—in the case of polyisocyanates-aliphatically attached isocyanate groups are preferably attached at the terminal carbon atoms of the carbon chain.

Preferred polyisocyanates having aliphatically attached isocyanate groups are 1,4-diisocyanatobutane (BDI), 1,5-diisocyanatopentane (PDI), 1,6-diisocyanatohexane (HDI), 2-methyl-1,5-diisocyanatopentane, 1,5-diisocyanato-2,2-dimethylpentane, 2,2,4- or 2,4,4-trimethyl-1,6-diisocyanatohexane, and 1,10-diisocyanatodecane.

In an isocyanate having cycloaliphatically attached isocyanate groups, all isocyanate groups are attached to carbon atoms that are part of a closed ring of carbon atoms. This ring may be unsaturated in one or more positions, provided it does not acquire aromatic character as a result of the presence of double bonds.

Preferred polyisocyanates having cycloaliphatically attached isocyanate groups are 1,3- and 1,4-diisocyanatocyclohexane, 1,4-diisocyanato-3,3,5-trimethylcyclohexane, 1,3-diisocyanato-2-methylcyclohexane, 1,3-diisocyanato-4-methylcyclohexane, 1-isocyanato-3,3,5-trimethyl-5-isocyanatomethylcyclohexane (isophorone diisocyanate; IPDI), 1-isocyanato-1-methyl-4 (3)-isocyanatomethylcyclohexane, 2,4′- and 4,4′-diisocyanatodicyclohexylmethane (H12MDI), 1,3- and 1,4-bis(isocyanatomethyl)cyclohexane, bis(isocyanatomethyl) norbornane (NBDI), 4,4′-diisocya-nato-3,3′-dimethyldicyclohexylmethane, 4,4′-diisocyanato-3,3′,5,5′-tetramethyldicyclohexyl-methane, 4,4′-diisocyanato-1,1′-bi(cyclohexyl), 4,4′-diisocyanato-3,3′-dimethyl-1,1′-bi(cyclohexyl), 4,4′-diisocyanato-2,2′,5,5′-tetramethyl-1,1′-bi(cyclohexyl), 1,8-diisocyanato-p-menthane, 1,3-diisocyanatoadamantane, and 1,3-dimethyl-5,7-diisocyanatoadamantane.

In an isocyanate having araliphatically attached isocyanate groups, all isocyanate groups are attached to methylene radicals that are in turn attached to an aromatic ring.

Preferred polyisocyanates having araliphatically attached isocyanate groups are 1,3- and 1,4-bis(isocyanatomethyl)benzene (xylylene diisocyanate; XDI), 1,3- and 1,4-bis(1-isocyanato-1-methylethyl)benzene (TMXDI), and bis(4-(1-isocyanato-1-methylethyl)phenyl) carbonate.

In an isocyanate having aromatically attached isocyanate groups, all isocyanate groups are attached directly to carbon atoms that are part of an aromatic ring.

Preferred isocyanates having aromatically attached isocyanate groups are tolylene diisocyanate (TDI), methylene diphenyl isocyanate (MDI) and naphthylene diisocyanate.

The term “tolylene diisocyanate” refers to tolylene 2,4-diisocyanate (2,4-TDI), tolylene 2,6-diisocyanate (2,6-TDI), and also any desired mixtures of the two isomers. The term “methylene diphenyl isocyanate” refers to all isomers of MDI, in particular diphenylmethane 2,2′-diisocyanate, diphenylmethane 2,4′-diisocyanate, diphenylmethane 4,4′-diisocyanate, all mixtures containing at least two of the aforementioned isomers, and also multiring derivatives of MDI.

The term “naphthylene diisocyanate” refers to naphthylene 1,4-diisocyanate, naphthylene 1,5-diisocyanate and naphthylene 1,6-diisocyanate, and also any desired mixtures of the aforementioned isomers.

It is known to those skilled in the art that all the abovementioned polyisocyanates may also be present as oligomers, and so the urethane linkage to be cleaved is formed between an oligomeric polyisocyanate, based on one of the abovementioned monomeric diisocyanates, and an alcohol.

The term “oligomeric polyisocyanate” refers to polyisocyanate composed of at least two of the abovementioned diisocyanates. The oligomeric polyisocyanates may, in accordance with the invention, especially have uretdione, isocyanurate, allophanate, biuret, iminooxadiazinedione and/or oxadiazinetrione structure. Preferably, the oligomeric polyisocyanates have at least one of the following oligomeric structure types or mixtures thereof:

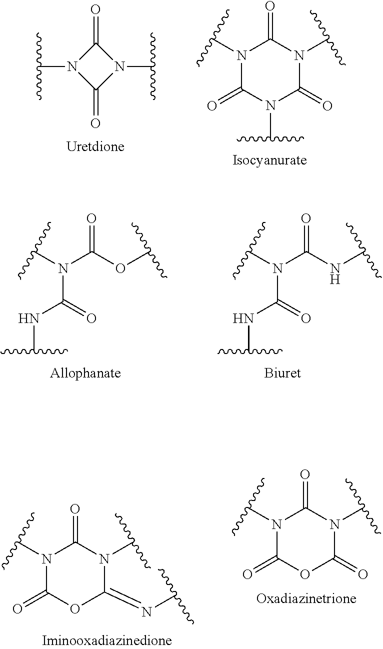 [SMILES: *N1C(=O)N(*)C1=O ; *n1c(=O)n(*)c(=O)n(*)c1=O ; *NC(=O)N(*)C(=O)O* ; *NC(=O)N(*)C(=O)N* ; */N=c1/oc(=O)n(*)c(=O)n1* ; *n1c(=O)oc(=O)n(*)c1=O]

Preferred substrates of the urethanase of the present invention are urethanes which can be obtained from the above-defined aromatic polyisocyanates.

### Enzymatic Cleavage

The expression “enzymatic cleavage of a urethane group” indicates that the cleavage of a urethane group described above proceeds more rapidly in the presence of a polypeptide having urethanase activity than it does when incubated with the reaction buffer without enzyme under the same reaction conditions or when incubated with the reaction buffer under the same conditions in the presence of an inactive polypeptide. The preferred model for an inactive polypeptide is bovine serum albumin. If, in the presence of a polypeptide being tested, the cleavage of the urethane group proceeds more rapidly than in an otherwise identical control with BSA, said polypeptide possesses urethane activity as understood in this application.

### Nucleic Acid

In a further embodiment, the present invention relates to a nucleic acid encoding a polypeptide as defined in SEQ ID NO.: 1, 2 or 3 or one of the above-defined variants of these polypeptides, wherein the encoded polypeptide has urethanase activity. Preferably, the coding nucleic acid sequence is under the control of a promoter which allows the expression of the polypeptide in a fungus, a yeast or a bacterium.

### Use

In a further embodiment, the present invention relates to the use of a polypeptide having an amino acid sequence as defined by SEQ ID NO.: 1, 2 and 3, or of one of the variants of these polypeptides defined above in this application, characterized in that the polypeptide has urethanase activity, for enzymatic cleavage of urethane linkages.

Unless explicitly defined otherwise, all definitions given above apply to this embodiment too. The use preferably consists in contacting the enzyme according to the invention with a compound containing at least one urethane linkage. This is preferably done under conditions where the enzyme shows its urethanase activity. Such conditions can be found in the working examples of this application. Furthermore, those skilled in the art are readily capable, by means of a simple series of experiments, of adjusting the reaction conditions in a system, for example temperature, ionic strength, solvent and pH, such that the enzyme is active.

For the urethanase according to the invention, the temperature during use is preferably in the range between 10° C. and 70° C. A higher temperature range between 30° C. and 70° C. is preferred here if the reaction rate is more important than the stability of the enzyme. Conversely, a temperature range between 10° C. and 40° C. is preferred if the reaction rate is of less importance.

When using the urethanases defined by SEQ ID NO. 1 and 2 and their variants, the pH is preferably between 7 and 12, more preferably between 7 and 11 and most preferably between 8 and 11. When using the urethanases defined by SEQ ID NO.: 3 or one of their variants, the pH is preferably between 7 and 12, more preferably between 7 and 11 and most preferably between 9 and 11.

### Cleavage of Low-Molecular-Weight Urethanes

In yet a further embodiment, the present invention relates to a process comprising the process step of treating a low-molecular-weight urethane with a polypeptide having urethanase activity and selected from the group consisting of a polypeptide defined by SEQ ID No. 1, 2 and 3 and the variants of these polypeptides defined above in this application, thereby cleaving the low-molecular-weight urethane.

In this context, the term “low-molecular-weight” means that the urethane contains not more than four, preferably not more than three and more preferably not more than one or two urethane groups.

Preferably, the structures of the low-molecular-weight urethanes are formally based on the formation of one or more urethane linkages between a polyisocyanate and one or more low-molecular-weight alcohols.

Here, preference is given to the polyisocyanates already mentioned further above as partners in the urethane linkages to be cleaved by the urethanases according to the invention. Particular preference is given here to aromatic polyisocyanates, in particular tolylene 2,4-diisocyanate (2,4-TDI), tolylene 2,6-diisocyanate (2,6-TDI), diphenylmethane 2,2′-diisocyanate (2,2′-MDI), diphenylmethane 2,4′-diisocyanate (2,4′-MDI), diphenylmethane 4,4′-diisocyanate (4,4′-MDI), the multiring derivatives of the aforementioned diphenylmethane diisocyanates, naphthylene 1,4-diisocyanate, naphthylene 1,5-diisocyanate and naphthylene 1,6-diisocyanate.

In principle, any compound having at least one hydroxyl group per molecule is suitable as a low-molecular-weight alcohol. It preferably has a molecular weight of not more than 700 g/mol, more preferably not more than 500 g/mol and most preferably not more than 200 g/mol.

Furthermore, preference is given to low-molecular-weight alcohols having relatively high polarity. Particularly preferably, the low-molecular-weight alcohol contains one or two hydroxyl groups per molecule. Furthermore, the low-molecular-weight alcohol preferably has a melting point of not more than 45° C., more preferably not more than 20° C.

Particular preference is therefore given to alcohols having two hydroxyl groups, a molecular weight of not more than 500 g/mol and a melting point of not more than 45° C.

In a particularly preferred embodiment of the present invention, the low-molecular-weight alcohol is selected from the group consisting of ethylene glycol, diethylene glycol, propylene glycol, dipropylene glycol, methyl glycol, triethylene glycol, glycerol, 2-methyl-1,3-propanediol, 1,4-butanediol, 1,5-pentanediol, 1,6-hexanediol and polyethylene glycol 400. It is more preferably selected from the group consisting of ethylene glycol, diethylene glycol, propylene glycol, dipropylene glycol, methyl glycol and triethylene glycol. Very particular preference is given to the low-molecular-weight alcohol diethylene glycol.

A low-molecular-weight urethane particularly preferred for the process according to the invention is obtainable by forming a urethane linkage between one of the low-molecular-weight alcohols mentioned in the preceding paragraph, in particular diethylene glycol, and.

The low-molecular-weight urethanes can be obtained in different ways. In a preferred embodiment of the present invention, the ester bonds of a polyester polyurethane are cleaved, so that carboxylic acids and low-molecular-weight urethanes are released.

In a preferred embodiment of the invention, the ester groups are preferably cleaved enzymatically. Preference is given here to using lipases (EC 3.1.1.3) or cutinases (EC 3.1.1.74).

In a further preferred embodiment of the present invention, preference is given to low-molecular-weight urethanes obtained by the chemical cleavage of a polyether polyurethane or polyester polyurethane. Said chemical cleavage is preferably a so-called “alcoholysis” or “glycolysis”, in which the polyether polyurethane or polyester polyurethane is reacted with one of the abovementioned low-molecular-weight alcohols, so that the ether component of the polyether polyurethane or the polyester polyol of a polyester polyurethane is replaced by the low-molecular-weight alcohol.

A particularly preferred embodiment for provision of low-molecular-weight urethanes from polyether polyurethanes is described in WO 2021/032513. In this process, polyether polyurethanes are reacted with a low-molecular-weight alcohol in such a way that the polyether component of the polyether polyurethane is released and a urethane linkage is formed between the low-molecular-weight alcohol used and the isocyanates used for synthesis of the polyether polyurethane. Thus the low-molecular-weight urethanes to be cleaved according to the invention are compounds which are formally obtained by forming a urethane linkage between a polyisocyanate, preferably one of the aliphatic, aromatic, araliphatic or cycloaliphatic polyisocyanates listed further above in this application, and a low-molecular-weight alcohol.

### Polyester Polyurethane

The term “polyester polyurethane” refers to a polyurethane, the polyol component of which contains polyester polyols. Preferably, at least 40% by weight of the hydroxyl groups present in the polyol component are constituents of polyester polyols. More preferably, this is at least 60% by weight, even more preferably at least 80% by weight, and most preferably at least 95% by weight. The polyester polyurethane may be foamed or nonfoamed.

The polyester polyurethane contains as the isocyanate component at least one aromatic, aliphatic or cycloaliphatic isocyanate. The polyurethane preferably comprises aromatic isocyanates. The at least one aromatic polyisocyanate is particularly preferably selected from the list given further above.

The term “polyester polyol” is known to those skilled in the art and describes polyesters containing an average of at least 1.5, preferably at least 1.8, and more preferably at least 2.0, hydroxyl groups per molecule. The polyester polyols present in the polyurethane to be broken down particularly preferably have functionality of between 1.5 and 6.0. They contain as structural elements aromatic and/or aliphatic polyols and also aromatic and/or aliphatic polycarboxylic acids in any combination. Polyester polyols obtained by ring-opening polymerization of a lactone are also preferred.

### Polyether Polyurethane

The term “polyether polyurethane” refers to a polyurethane, the polyol component of which contains polyether polyols. Preferably, at least 40% by weight of the hydroxyl groups present in the polyol component are constituents of polyether polyols. More preferably, this is at least 60% by weight, even more preferably at least 80% by weight, and most preferably at least 95% by weight. It is in accordance with the invention possible for a polyether polyurethane to also contain further polyols as structural components while maintaining the abovementioned proportions of polyether polyols. These are preferably polyester polyols.

The term “polyether polyol” is well known to those skilled in the art. These are polyethers having an average hydroxyl functionality of between 1.5 and 6.0. The polyether polyol present in the polyether polyurethane is preferably a polyaddition product of one or more alkylene oxides having 2 to 4 carbon atoms using at least one starter molecule containing 2 to 8, preferably 2 to 6, attached reactive hydrogen atoms.

Preferred alkylene oxides are styrene oxide, ethylene oxide, propylene oxide, tetrahydrofuran, butylene oxide, and epichlorohydrin. Greater preference is given to 1,3-propylene oxide, 1,2- or 2,3-butylene oxide, and styrene oxide. Particular preference is given to ethylene oxide and 1,2-propylene oxide. The alkylene oxides may be used individually, alternately in succession or as mixtures.

The “treatment” of the low-molecular-weight urethane is done under conditions where the enzyme used shows urethanase activity. Under these conditions, the reaction products of the enzymatic cleavage of the low-molecular-weight urethane that are formed are a low-molecular-weight alcohol or a mixture of low-molecular-weight alcohols and an amine or a mixture of amines. The chemical structure of the amines formed depends on the nature of the polyisocyanate used to synthesize the urethane. The amines liberated are amines which can be derived from the polyisocyanates used by addition of water and subsequent elimination of CO₂.

In yet a further embodiment, the present invention relates to the use of at least one of the polypeptides according to the invention for cleavage of a urethane linkage in low-molecular-weight urethanes.

Breakdown of Polyester Urethanes into Low-Molecular-Weight Breakdown Products

In yet a further embodiment, the present invention relates to a process for breaking down polyester polyurethanes into low-molecular-weight breakdown products, comprising the steps of
a) cleaving the ester groups present in the polyester polyurethane; and
b) treating the polyurethane with a polypeptide having urethanase activity and selected from the group consisting of polypeptides as defined in SEQ ID NO.: 1, 2 and 3 and the variants of said polypeptides defined above in this application;
   with the proviso that process steps a) and b) may be carried out in either order or else in parallel.

Preference is given to carrying out process step a) before process step b).

Unless otherwise stated, all definitions given further above in this application also apply to this embodiment. This applies in particular to the treatment of the polyurethane with a urethanase in process step b).

In a preferred embodiment of the invention, process step a) is carried out with a lipase or cutinase. Particular preference is given to using a lipase capable of cleaving tributyrin. Process step a) is preferably carried out under reaction conditions where the lipase or cutinase used shows activity. Such conditions can be determined by routine experiments using common biochemical methods.

In another embodiment that is also preferred, process step a) is carried out chemically.

The polyurethane comprises as the isocyanate component at least one aromatic, aliphatic or cycloaliphatic isocyanate. The polyurethane preferably comprises aromatic isocyanates.

The low-molecular-weight breakdown products of the polyester-based polyurethanes preferably have a molecular weight of not more than 1000 g/mol. These are preferably
(i) amines derived from the isocyanates used in the production of the polyurethane concerned, for example tolylene-2,4-diamine in the case of tolylene 2,4-diisocyanate; and
(ii) alcohols and carboxylic acids used to form the polyester polyols employed in the synthesis of the polyurethane concerned.

A “polyol” formed as a low-molecular-weight breakdown product in the process defined in this section is understood in this application to mean any compound having at least two hydroxyl groups. Said polyol preferably has a molecular weight of not more than 300 g/mol. Preferred polyols that are low-molecular-weight breakdown products of the polyester-based polyurethanes are selected from the group consisting of ethylene glycol, diethylene glycol, triethylene glycol, propylene glycol, 1,3-pentanediol, 1,4-butanediol, 1,6,-hexanediol, 1,2-dipropylene glycol, neopentyl glycol, glycerol, 1,1,1-trimethylolpropane, sucrose, sorbitol, and pentaerythritol.

A “polycarboxylic acid” formed as a low-molecular-weight breakdown product in the process defined in this section is understood in this application to mean any compound containing at least two carboxyl groups. Said polycarboxylic acid preferably has a molecular weight of not more than 300 g/mol. Preferred polycarboxylic acids that are low-molecular-weight breakdown products of the polyester-based polyurethane foams are selected from the group consisting of succinic acid, glutaric acid, adipic acid, itaconic acid, phthalic acid, isophthalic acid, terephthalic acid, and benzenetricarboxylic acid.

A “polyamine” formed as a low-molecular-weight breakdown product in the process defined in this section is understood in this application to mean any compound containing at least two amino groups. Said polyamine preferably has a molecular weight of not more than 300 g/mol. Preferred polyamines that are low-molecular-weight breakdown products of the polyester-based polyurethane foams are selected from the group consisting of 4,4′-diaminodiphenylmethane, 2,4′-diaminodiphenylmethane, 2,2′-diaminodiphenylmethane, the multiring derivatives of the aforementioned diaminodiphenylmethane compounds, tolylene-2,4-diamine, tolylene-2,6-diamine, naphthylene-1,4-diamine, naphthylene-1,5-diamine, naphthylene-1,6-diamine, hexamethylenediamine, isophorone diamine, xylenediamine, pentamethylenediamine, para-phenylenediamine, butyldiamine, and H12-methylenediamine. Particular preference is given to polyamines selected from the group consisting of 4,4′-diaminodiphenylmethane, 2,4′-diaminodiphenylmethane, 2,2′-diaminodiphenylmethane, naphthylene-1,5-diamine, tolylene-2,4-diamine, and tolylene-2,6-diamine.

### Breakdown of Polyether Polyurethane

In yet a further embodiment, the present invention relates to a process comprising the steps of
a) transurethanizing a polyether polyurethane with at least one low-molecular-weight alcohol to form polyether polyols and low-molecular-weight urethanes; and
b) enzymatically cleaving the low-molecular-weight urethanes formed in process step a) with a polypeptide having urethanase activity and selected from the group consisting of polypeptides as defined in SEQ ID NO.: 1, 2 and 3 and the variants of said polypeptides defined above in this application.

Process step a) has two aims: (i) The polyether polyol used to synthesize the polyurethane should be liberated from the polyurethane as an isolable compound. (ii) The isocyanate used to synthesize the polyurethane should be present as a constituent of a low-molecular-weight urethane. In contrast to polyurethane with its high molecular weight, said low-molecular-weight urethane is on account of its lower molecular weight and consequent better solubility well suited as a substrate for the enzymatic cleavage that takes place in process step b).

Unless otherwise stated, all definitions given further above in this application also apply to this embodiment. This applies in particular to the enzymatic cleavage of the low-molecular-weight urethanes in process step b).

In process step b), an amine and the low-molecular-weight alcohol used for transurethanization in process step a) are liberated by enzymatic cleavage of low-molecular-weight urethanes. In addition, CO₂ is evolved in this process step. These compounds can be isolated by suitable methods of separation and then used further. Here, it is preferable for the liberated low-molecular-weight alcohol to be reused for the transurethanization that takes place in process step a). The liberated amine is available as a pure and well-defined starting material for new syntheses.

The reaction conditions and catalysts suitable in principle for the transurethanization in process step a) are described in Simon et al. (2018), Waste Management, 76:147-171. Process step a) is carried out at temperatures of between 140° C. and 300° C., preferably of between 160° C. and 270° C. The weight ratio of the low-molecular-weight alcohol to the polyether urethane is between 2:1 and 1:17. Particularly suitable as catalysts are alkali metal hydroxides, alkaline earth metal hydroxides, alkali metal salts of carboxylic acids (in particular acetates), alkaline earth metal salts of carboxylic acid (in particular acetates), Lewis acids (such as in particular dibutyltin dilaurate), organic amines (such as in particular diethanolamine), organometallic compounds (such as in particular titanium tetrabutoxide), and tin compounds (such as in particular tin octoate). The transurethanization is preferably carried out at temperatures in the range from 160° C. to 270° C. in the presence of 0.1% by mass to 5% by mass of catalyst, based on the mass of the polyurethane product added.

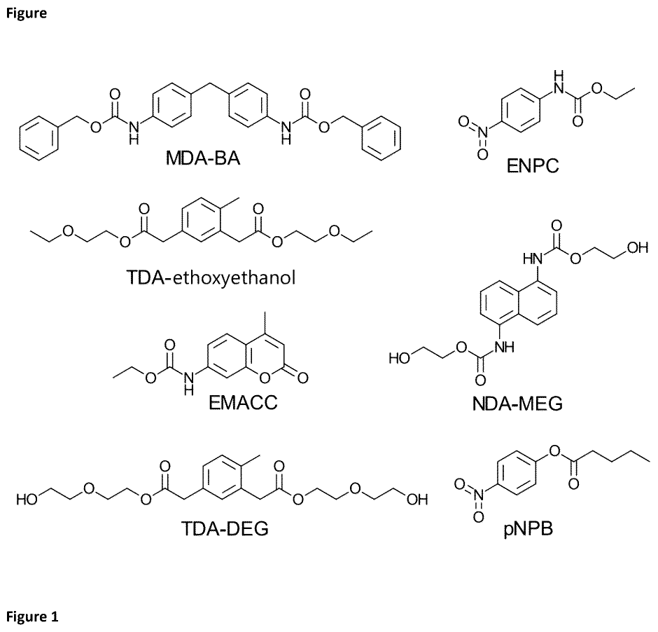

FIG. 1 shows: Enzyme substrates used. pNPB: 4-nitrophenyl butyrate; ENPC: ethyl 4-nitrophenyl carbamate; MDA-BA: carbamate of 4,4′-MDI reacted with benzyl alcohol; TDA-ethoxyethanol: carbamate of toluene diisocyanate (TDI) reacted with ethoxyethanol; TDA-DEG: carbamate of TDI reacted with diethylene glycol; NDA-MEG: carbamate of NDI reacted with ethylene glycol; EMACC: carbamate from the reaction of 7-amino-4-methylcoumarin with ethyl chloroformate.

The working examples that follow serve merely to elucidate the invention. They are not intended to limit the scope of the claims in any way.

### EXAMPLES

### Example 1: Determination of Activity Using EMACC

### Enzyme Preparation

Urethanase genes were cloned into the pET-26 expression vector directly after the NdeI cleavage site. For expression, E. coli BL21 (DE3) were transformed with the relevant plasmids. A single colony was used for inoculation of the overnight culture in LB medium containing 1% (w/v) glucose and 50 μg/mL kanamycin. For the main culture, 200 ml of ZYP-5052 containing 50 μg/mL kanamycin were inoculated with 1 mL of the overnight culture and incubated in baffled flasks at 37° C. at 100 rpm for 4 h. The temperature was then reduced to 20° C. before the cultures were harvested by centrifugation at 4500 g and 4° C. for 20 min. Cell pellets were stored at −20° C. before the proteins were purified.

For purification, 15 mL of lysis buffer (50 mM sodium phosphate (NaPi), pH 8.0, 300 mM NaCl, 10 mM imidazole) were added to the cell pellet. Disruption was carried out by ultrasound on ice for two cycles (3 min, 50% pulse, 50% power). The lysate was cleared by centrifugation at 10 000 g and 4° C. for 40-60 min. The supernatants were stored on ice before the IMAC column was loaded. IMAC resin (1 ml, ROTI® Garose His Beads, nickel form) was equilibrated with lysis buffer. The lysate was then applied to the columns followed by a wash step with at least ten column volumes of wash buffer (50 mM NaPi, pH 8.0, 300 mM NaCl, 20 mM imidazole). The proteins were then eluted with 15 mL of elution buffer (50 mM NaPi, pH 8.0, 300 mM NaCl, 250 mM imidazole). The volume of the elution fraction was reduced to 2.5 mL using Vivaspin 20 ultrafiltration units (10 kDa MWCO). The proteins were then rebuffered into storage buffer (10 mM NaPi, pH 8.0, 100 mM NaCl) using PD10 columns.

### EMACC Assay

To determine urethanase activity, a fluorogenic substrate was synthesized from 7-amino-4-methylcoumarin (AMC) and ethyl chloroformate. The ethyl carbamate of AMC thus formed (EMACC) was in the form of a fine powder. Specific activity was determined under standard conditions (50 mM NaPi, pH 8.0, 30° C., 100 UM EMACC). EMACC was freshly added from a 50 mM DMSO stock. Purified urethanases were diluted in storage buffer before addition to the assay, such that there was a linear increase in absorption at 365 nm over 10-30 min. Measurement was carried out in 96-well plates in a plate photometer preheated to 30° C. and specific activity was calculated from the linear increase in absorption in the first few minutes. The results are shown in table 1.

TABLE 1
Specific activity of urethanase with respect to EMACC

| | Specific activity toward EMACC |
|---|---|
| Urethanase | [μmol/min/mg enzyme] |
| UMG-SP-8 | 0.749 ± 0.012 |
| (SEQ ID NO.: 1) | |
| UMG-SP-11.2 | 0.377 ± 0.020 |
| (SEQ ID NO.: 2) | |
| UMG-SP-16 | 0.651 ± 0.064 |
| (SEQ ID NO.: 3) | |

### Example 2: Screening Against Further Substrates

Enzyme Preparation To prepare the enzyme preparations, single colonies of E. coli BL21 (DE3) pET26b_UMG-8, BL21 (DE3) pET26b_UMG-11.2 and BL21 (DE3) pET26b_UMG-16 were each used to inoculate 4.5 mL of LB medium containing 1% (w/v) glucose, and they were incubated overnight in an incubation shaker at 37° C. and 200 rpm. All the cultures were admixed with 50 μg/mL kanamycin. Thereafter, 200 μL of each overnight culture was used to inoculate 200 ml of ZYP-5052 medium. Cultivation was carried out in an incubation shaker for 4 h at 37° C. and 200 rpm, followed by 24 h at 20° C. and 200 rpm. The cells were removed by centrifugation in a high-capacity centrifuge at 20 425 g and 4° C. for 20 min. The cell pellets were each suspended in 6 ml of disruption buffer (20 mM ammonium acetate, 0.4% n-dodecyl-β-maltoside, 1% lysozyme, 1 L/mL benzonase (Sigma-Aldrich Chemie GmbH, Taufkirchen)) and incubated on an orbital shaker at room temperature for 30 min. The total cell extract solution was then frozen at −80° C. and lyophilized. The lyophilizate was stored at 4° C.

### Enzyme Reactions

The substrates ENPC, MDA-BA, NDA-MEG, TDA-ethoxyethanol and TDA-DEG were synthesized from the corresponding isocyanates and alcohols by reacting the isocyanates with an excess of alcohol. EMACC and 4-nitrophenyl butyrate (pNPB) were used to confirm urethanase activity. The enzyme substrates are shown in FIG. 1.

One spatula tip (˜3 mg) each of the lyophilized total cell extracts was suspended in 800 μL of 100 mM KPi containing 100 mM NaCl, pH 7.5. The enzyme solution was diluted 1:10 in the reaction mixture containing the same buffer. Substrate stocks of the carbamates were prepared in DMSO at a concentration of 5-340 mg/mL. These were diluted 1:10 in the reaction mixture, such that the final DMSO concentration was 10% (v/v). The reactions were prepared with a total volume of 500 μL. Incubation was carried out in a thermal shaker for one day at 30° C. and 800-1000 rpm, followed by one day at 40° C. and 800-1000 rpm. Following incubation, 500 μL of acetonitrile were added to improve the solubility of substrates and products. The samples were filtered through a 0.2 μm PVDF filter plate by centrifugation and used for HPLC analysis. For the substrates EMACC and pNPB, the total volume of the reaction was 200 μL, and fluorescence (EMACC, excitation at 365 nm, emission at 440 nm) or absorption (pNPB, 410 nm) was measured at various times in a Varioskan Lux plate photometer (Thermo Fisher Scientific Inc., Waltham, Massachusetts, USA).

### HPLC Analysis

High-pressure liquid chromatography was carried out on an 1260 Infinity II series instrument from Agilent Technologies (Santa Clara, USA), equipped with a multisampler and DAD (diode array detector) for UV and the visible light region. All measurements were carried out using a “Zorbax Eclipse Plus C18” column having a particle size of 5 μm and dimensions of 4.6×150 mm (Agilent Technologies, Santa Clara, USA) with an appropriate precolumn. In all methods, a 5 μL sample was injected into a column heated to 40° C. The flow was generally 1.0 mL/min.

Aromatic amines were detected by using the “ER_Juni2021-2” method. This method allowed the quantification of aromatic amines at 210 nm and 232 nm without derivatization on account of their high intrinsic absorption. ddH₂O containing 5% (v/v) acetonitrile was used as eluent A, and acetonitrile containing 5% (v/v) ddH₂O was used as eluent B. The data were analyzed using the “OpenLAB CDS 2.4” software, version 2.204.0.661 (Agilent Technologies, Santa Clara, USA).

TABLE 2
HPLC gradient

| t [min] | Eluent A [%] | Eluent B [%] |
|---|---|---|
| 0.00 | 100.00 | 0.00 |
| 2.00 | 100.00 | 0.00 |
| 10.00 | 0.00 | 100.00 |
| 11.00 | 0.00 | 100.00 |
| 11.50 | 100.00 | 0.00 |
| 15.00 | 100.00 | 0.00 |

### Results

The hydrolytic activity of the urethanases with respect to the carbamates tested was classified into the categories “no activity” (−), “little activity” (+), “medium activity” (+) and “high activity” (+) on the basis of the peak area in the chromatogram of the products formed. It was found that the urethanases UMG-8, UMG11.2 and UMG 16 accept a broad spectrum of substrates. The substrates tested are shown in FIG. 1.

TABLE 3
Urethanase activity with respect to various carbamates.

| | UMG-8 | UMG-11.2 | UMG-16 |
|---|---|---|---|
| | (SEQ ID | (SEQ ID | (SEQ ID |
| Substrate | NO.: 1) | NO.: 2) | NO.: 3) |
| EMACC | +++ | +++ | +++ |
| pNPB | +++ | +++ | +++ |
| ENPC | ++ | +++ | ++ |
| MDA-BA | ++ | ++ | + |
| NDA-MEG | ++/+++ | ++/+++ | ++ |
| TDA-ethoxyethanol | +++ | +++ | +++ |
| TDA-DEG | ++ | +++ | ++ |

Activity is divided into the categories “no activity” (−), “little activity” (+), “medium activity” (++) and “high activity” (+++) on the basis of HPLC peak area.

### Example 3: Optimization of Reaction Conditions

To determine pH optimums, enzyme activity was determined analogously to example 1, with the modification of carrying out the reaction in different buffers (100 mM). The buffers used were buffers of pH 4.0, 5.0 and 6.0 (citrate), 7.0, 8.0 and 9.0 (bis-tris propane) and 10.0 (CHES). In the case of pH values 10, 11, 12 and 13, the buffer used was a buffer composed of 100 mM sodium phosphate and 100 mM sodium carbonate and—as a departure from example 1—containing 200 mM EMACC and 10% (v/v) DMSO.

Temperature optimums were determined by measuring the release of AMC from 100 μM EMACC in 50 mM NaPi (pH 8.0) at 30° C., 35° C., 40° C., 45° C., 50° C., 55° C., 60° C., 65° C. and 70° C. Solvent stability was determined by quantifying the hydrolysis of EMAC under the conditions specified in example 1 at 0%, 10%, 20%, 30%, 40% and 50% (v/v) DMSO. The activity optimums determined herein for pH, temperature and DMSO concentration are

| | pH | T | Optimal DMSO |
|---|---|---|---|
| Urethanase | optimum | optimum (° C.) | concentration (% v/v) |
| UMG-SP-8 | 10 | 70 | 0 |
| (SEQ ID NO.: 1) | | | |
| UMG-SP-11.2 | 10 | 70 | 10 |
| (SEQ ID NO.: 2) | | | |
| UMG-SP-16 | 10 | 35 | 10 |
| (SEQ ID NO.: 3) | | | |

summarized in table 4. The dependence of the activity is in

TABLE 4
Optimal reaction conditions for urethanase.

| | pH | T | Optimal DMSO |
|---|---|---|---|
| Urethanase | optimum | optimum (° C.) | concentration (% v/v) |
| UMG-SP-8 | 10 | 70 | 0 |
| (SEQ ID NO.: 1) | | | |
| UMG-SP-11.2 | 10 | 70 | 10 |
| (SEQ ID NO.: 2) | | | |
| UMG-SP-16 | 10 | 35 | 10 |
| (SEQ ID NO.: 3) | | | |

TABLE 5
pH dependence of urethanase activity. In both data sets,
activity was highest at pH 10, and so normalization was
carried out to this value. The activity data of pH 12
and pH 13 were not shown because the fluorescence of
AMC is constant only in the range between pH 3 and pH 12.

| | pH | | | | | | | |
|---|---|---|---|---|---|---|---|---|
| | 4 | 5 | 6 | 7 | 8 | 9 | 10 | 11 |
| UMG-SP-8 | 0% | 0% | 0% | 41% | 77% | 82% | 100% | 86% |
| (SEQ ID No. 1) | | | | | | | | |
| UMG-SP-11.2 | 0% | 0% | 0% | 5% | 15% | 25% | 100% | 20% |
| (SEQ ID NO.: 2) | | | | | | | | |
| UMG-SP-16 | 0% | 0% | 0% | 5% | 7% | 11% | 100% | 34% |
| (SEQ ID NO.: 3) | | | | | | | | |

### Temperature Stability

To determine long-term temperature stability, solutions of the purified urethanases having a protein concentration of 0.5 mg/ml in 100 mM KPi, pH 7.5, were prepared. 100 μL in each case were incubated in a Biometra TAdvanced Basis thermal cycler (Analytik Jena GmbH, Jena) at 20° C., 25.7° C., 29.4° C., 36.9° C., 40.6° C., 44.3° C. and 50° C. for 12 h and then at 4° C. before measurement of activity. The temperature of the lid heater was adjusted to 99° C. A reference sample was incubated at 4° C. The residual activity of the enzymes was determined using a pNPB assay. To this end, 21 μL of pNPB were mixed with 4.979 mL of DMSO. This solution was diluted 1:10 in 100 mM KPi, pH 7.5. 20 μL of enzyme solution (UMG-8 diluted 1:5 in 100 mM KPi, PH 7.5, UMG-11.2 diluted 1:20 in 100 mM KPi, pH 7.5, UMG-16 diluted 1:20 in 100 mM KPi, pH 7.5) were loaded in a microplate, and the reaction was started by adding 180 μL of pNPB in 100 mM KPi, pH 7.5, containing 10% DMSO. When the pNPB is hydrolyzed by the urethanases, para-nitrophenol is released and this was quantified by measuring the absorption at 410 nm in a Varioskan Lux plate reader (Thermo Fisher Scientific Inc., Waltham, Massachusetts, USA). The results are shown in table 6.

TABLE 6
Specific urethanase activity with respect to pNPB in U/mg following incubation for 12 h at 20°
C., 25.7° C., 29.4° C., 36.9° C., 40.6° C., 44.3° C. and 50° C. and at 4° C. as reference.

| | Incubation temperature | | | | | | | |
|---|---|---|---|---|---|---|---|---|
| | 4° C. | 20° C. | 25.7° C. | 29.4° C. | 36.9° C. | 40.6° C. | 44.3° C. | 50° C. |
| Enzyme | Specific activity [μmol/min/mg enzyme] | | | | | | | |
| UMG-8 | 6.73 | 6.50 | 7.12 | 6.42 | 3.23 | 0 | 0 | 0 |
| (SEQ ID NO.: 1) | | | | | | | | |
| UMG-SP-11.2 | 22.81 | 8.51 | 21.44 | 10.00 | 0 | 0 | 0 | 0 |
| (SEQ ID NO.: 2) | | | | | | | | |
| UMG-SP-16 | 27.86 | 0 | 0 | 0 | 0 | 0 | 0 | 0 |
| (SEQ ID NO.: 3) | | | | | | | | |

1. A polypeptide having an amino acid sequence as defined by SEQ ID NO.: 1, 2 or 3, or a variant thereof,
wherein the variant is obtained by the addition, deletion or exchange of up to 15% of the amino acids present in the respective polypeptide defined by SEQ ID NO.: 1, 2 or 3, and
wherein the polypeptide has urethanase activity.

2. A method of breaking down a polyurethane, comprising enzymatically cleaving urethane linkages with a polypeptide having an amino acid sequence as defined by SEQ ID NO.: 1, 2 or 3, or a variant thereof,
wherein the variant is obtained by the addition, deletion or exchange of up to 15% of the amino acids present in the respective polypeptide defined by SEQ ID NO.: 1, 2 or 3, and
wherein the polypeptide has urethanase activity.

3. The method as claimed in claim 2, wherein the urethane linkage is aromatically attached.

4. A nucleic acid encoding a polypeptide as defined by SEQ ID NO. 1, 2 or 3 or a variant thereof having urethanase activity,
wherein the variant is obtained by the addition, deletion or exchange of up to 15% of the amino acids present in the respective polypeptide defined by SEQ ID NO.: 1, 2 or 3.

5. A process comprising treating a low-molecular-weight urethane with a polypeptide having urethanase activity and having an amino acid sequence as defined by SEQ ID No. 1, 2 or 3, or a variant thereof,
wherein the variant is obtained by the addition, deletion or exchange of up to 15% of the amino acids present in the respective polypeptide defined by SEQ ID NO.: 1, 2 or 3,
thereby cleaving the low-molecular-weight urethane.

6. The process as claimed in claim 5, wherein at least one polyamine selected from the group consisting of 4,4′-diaminodiphenylmethane, 2,4′-diaminodiphenylmethane, 2,2′-diaminodiphenylmethane, a multiring derivative of 4,4′-diaminodiphenylmethane, a multiring derivative of 2,4′-diaminodiphenylmethane, a multiring derivative of 2,2′-diaminodiphenylmethane, tolylene-2,4-diamine, tolylene-2,6-diamine, naphthylene-1,4-diamine, naphthylene-1,5-diamine and tolylene-2,6-diamine is formed.

7. The process as claimed in claim 5, wherein an alcohol having at least two hydroxyl groups and a molecular weight of not more than 700 g/mol is released.

8. The process as claimed in claim 7, wherein at least one alcohol selected from the group consisting of ethylene glycol, diethylene glycol, propylene glycol, dipropylene glycol, methyl glycol, triethylene glycol, glycerol, 2-methyl-1,3-propanediol, 1,4-butanediol, 1,5-pentanediol, 1,6-hexanediol and polyethylene glycol 400 is released.

9. A method of breaking down a low molecular weight urethane, comprising cleaving a urethane linkage with at least one polypeptide having urethanase activity and having an amino acid sequence as defined by SEQ ID NO.: 1, 2 or 3 or having an amino acid sequence with at least 85% sequence identity thereto.

10. A process for breaking down a polyester polyurethane into low-molecular-weight breakdown products, comprising:
a) cleaving the ester groups present in the polyester polyurethane; and
b) treating the polyurethane with a polypeptide having urethanase activity and selected from the group consisting of polypeptides as defined by SEQ ID NO.: 1, 2 and 3 and variants of said polypeptides,
wherein the variants are obtained by the addition, deletion or exchange of up to 15% of the amino acids present in the respective polypeptide defined by SEQ ID NO.: 1, 2 or 3; and
with the proviso that process steps a) and b) are carried out in either order or in parallel.

11. A process comprising:
a) transurethanizing a polyether polyurethane with at least one low-molecular-weight alcohol to form polyether polyols and low-molecular-weight urethanes; and
b) enzymatically cleaving the low-molecular-weight urethanes formed in process step a) with a polypeptide having urethanase activity and selected from the group consisting of polypeptides as defined by SEQ ID NO.: 1, 2 and 3 and variants of said polypeptides,
wherein the variants are obtained by the addition, deletion or exchange of up to 15% of the amino acids present in the respective polypeptide defined by SEQ ID NO.: 1, 2 or 3.
